(19) United States
(12) Patent Application Publication
(10) Pub. No.: US 2023/0159411 A1
(43) Pub. Date: May 25, 2023

(54) INTEGRATED PROCESS FOR PRODUCING ETHYLENE AND PROPYLENE FROM C4 AND/OR C5 HYDROCARBONS

(71) Applicant: UOP LLC, Des Plaines, IL (US)

(72) Inventors: Zhengjie Yao, Nanjing City (CN); Zhihao Fei, Naperville, IL (US); Lev Davydov, Northbrook, IL (US); Scott Lyle Nauert, Chicago, IL (US); Lubo Zhou, Deer Park, IL (US)

(21) Appl. No.: 17/675,969

(22) Filed: Feb. 18, 2022

(30) Foreign Application Priority Data
Nov. 25, 2021 (CN) 202111411989.1

(51) Int. Cl. C07C 4/06; C07C 7/00

(52) U.S. Cl. CPC C07C 4/06; C07C 7/005

(57) ABSTRACT

A process for production of light olefins by cracking a feed comprising C4 and/or C5 hydrocarbons. The process combines a catalytic naphtha cracking unit with an olefin paraffin separation unit and/or an olefin cracking unit. Contacting a feed stream comprising C4 and/or C5 olefins with a catalyst at catalytic naphtha cracking conditions produces a cracked product of ethylene and propylene.

### CROSS-REFERENCE TO RELATED APPLICATIONS

This application claims priority from Chinese Application No. 202111411989.1 filed Nov. 25, 2021, which is incorporated herein in its entirety.

### FIELD

The field is the production of light olefins by cracking a feed comprising C4 and/or C5 hydrocarbons.

### BACKGROUND

Light olefin production is vital to the production of sufficient plastics to meet worldwide demand. Ethylene and propylene are important chemicals for use in the production of other useful materials, such as polyethylene and polypropylene. Polyethylene and polypropylene are two of the most common plastics found in use today and have a wide variety of uses. Uses for ethylene and propylene include the production of vinyl chloride, ethylene oxide, ethylbenzene and alcohol.

The great bulk of the ethylene consumed in the production of plastics and petrochemicals such as polyethylene is produced by the thermal cracking of hydrocarbons. Steam is usually mixed with the feed stream to the cracking furnace to reduce the hydrocarbon partial pressure and enhance olefin yield and to reduce the formation and deposition of carbonaceous material in the cracking reactors. The process is therefore often referred to a steam cracking or pyrolysis.

Typically, one or more cracking feed streams may be preheated and fed to the steam cracking furnace for cracking of hydrocarbons under steam in the absence of catalyst to produce a plurality of cracked streams. The steam cracking furnace may be operated at a temperature of about 750° C. (1382° F.) to about 950° C. (1742° F.). The cracking feed streams may enter at the same point of the furnace, or at separate points to maximize product yields. Paraffinic feed streams, preferably ethane or predominantly normal paraffinic feed streams in the naphtha boiling range are typically preferred feed streams to pyrolysis.

The cracked stream exiting the furnace of the steam cracking unit may be in a superheated state. One or more quench columns, or other devices known in the art, may be used for quenching or separating the cracked stream into a plurality of cracked streams.

Typically, about 40 wt% of the products recovered are valuable light olefins. A polymerization plant may be on site, or the recovered olefins may be transported to a polymerization plant for polymer production.

Alternatively, catalytic cracking processes may be used to produce propylene and ethylene in addition to the predominant gasoline boiling range hydrocarbons. A commonly used catalytic cracking process is typically referred to as fluid catalytic cracking (FCC). The basic equipment or apparatus for the FCC of hydrocarbons has been in existence since the early 1940’s. The basic components of the FCC process include a reactor, a regenerator and a catalyst stripper. The reactor includes a contact zone where the hydrocarbon feed is contacted with a particulate catalyst and a separation zone where product vapors including propylene and ethylene from the cracking reaction are separated from the FCC catalyst. Further product separation takes place in a catalyst stripper that receives catalyst from the separation zone and removes entrained hydrocarbons from the catalyst by counter-current contact with steam or another stripping medium. The contact zone is typically a vertical fluidized reaction vessel where catalyst and hydrocarbon feed are co-currently rising. The FCC process is carried out by contacting the starting material whether it be vacuum gas oil, reduced crude, or another source of relatively high boiling hydrocarbons with a catalyst made up of a finely divided or particulate solid material. The catalyst is transported like a fluid by passing gas or vapor through it at sufficient velocity to produce a desired regime of fluid transport. Contact of the oil with the fluidized material catalyzes the cracking reaction and deposits coke on the catalyst. Coke is comprised of hydrogen and carbon and can include other materials in trace quantities such as sulfur and metals that enter the process with the starting feed. Coke interferes with the catalytic activity of the catalyst by blocking active sites on the catalyst surface where the cracking reactions take place.

Catalyst is traditionally transferred from the stripper to a regenerator for purposes of removing the coke by oxidation with an oxygen-containing gas. An inventory of catalyst having a reduced coke content, relative to the catalyst in the stripper, hereinafter referred to as regenerated catalyst, is collected for return to the reaction zone. Oxidizing the coke from the catalyst surface releases a large amount of heat, a portion of which escapes the regenerator with gaseous products of coke oxidation generally referred to as flue gas. The balance of the heat leaves the regenerator with the regenerated catalyst. The fluidized catalyst is continuously circulated from the reaction zone to the regeneration zone and then again to the reaction zone. The fluidized catalyst, as well as providing a catalytic function, acts as a vehicle for the transfer of heat from zone to zone. Catalyst exiting the reaction zone is spoken of as being spent, i.e., partially deactivated by the deposition of coke upon the catalyst.

The traditional steam cracking process still has drawbacks including low yields to ethylene and propylene yields, low selectivity to propylene, high reaction temperatures resulting in high energy consumption, and limited interest of certain governments in a new steam cracker project. Traditional FCC processes reduce reaction temperatures and therefore decrease energy consumption, but do not yet provide high enough light olefin yields. Thus, there is a need for a new integrated process without steam cracking to convert refinery C4, C5 stream and/or C6 to light olefins without steam cracking.

### BRIEF SUMMARY

We have combined a catalytic naphtha cracking unit with an olefin paraffin separation unit and/or an olefin cracking unit. Contacting a feed stream comprising C₄ and/or C₅ olefins with a catalyst at catalytic naphtha cracking conditions can produce a cracked product of ethylene and propylene.

### BRIEF DESCRIPTION OF THE DRAWINGS

FIG. 1 is a schematic drawing of a process of the present disclosure.

FIG. 2 is a schematic drawing of an alternate process of the present disclosure.

FIG. 3 is a schematic drawing of an alternate process of the present disclosure.

### DEFINITIONS

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

The term “upstream communication” means that at least a portion of the fluid flowing from the subject in upstream communication may operatively flow to the object with which it fluidly communicates.

The term “direct communication” means that fluid flow from the upstream component enters the downstream component without passing through any other intervening vessel.

The term “indirect communication” means that fluid flow from the upstream component enters the downstream component after passing through an intervening vessel.

The term “bypass” means that the object is out of downstream communication with a bypassing subject at least to the extent of bypassing.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripper columns may omit a reboiler at a bottom of the column and instead provide heating requirements and separation impetus from a fluidized inert media such as steam. Stripping columns typically feed a top tray and take main product from the bottom.

As used herein, the term “a component-rich stream” or “a stream rich in a component” means that the rich stream coming out of a vessel has a greater concentration of the component than any other stream from the vessel.

As used herein, the term “a component-lean stream” or “a stream lean in a component” means that the lean stream coming out of a vessel has a smaller concentration of the component than any other stream from the vessel.

As used herein, the term “initial boiling point” (IBP) means the temperature at which the sample begins to boil using ASTM D-7169, ASTM D-86 or TBP, as the case may be.

As used herein, the term “end point” (EP) means the temperature at which the sample has all boiled off using ASTM D-7169, ASTM D-86 or TBP, as the case may be.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure.

As used herein, the term “predominant” or “predominate” means greater than 50%, suitably greater than 75% and preferably greater than 90%.

The term “Cx” is to be understood to refer to molecules having the number of carbon atoms represented by the subscript “x”. Similarly, the term “Cx-” refers to molecules that contain less than or equal to x and preferably x and less carbon atoms. The term “Cx+” refers to molecules with more than or equal to x and preferably x and more carbon atoms.

The term “unit” is to be understood to refer to one or more process steps comprising a chemical transformation. At the heart of a unit is one or more catalytic reactors or separation vessels necessary to accomplish the transformation. A unit may further comprise additional separation steps known in the art to separate product streams. A unit may further comprise pretreatment steps known in the art for the chemical transformation. Taken together, “unit” comprises one or more reactors or separation vessels and separation steps and pretreatment steps, whether or not shown in the diagram or explicitly discussed in the specification.

### DETAILED DESCRIPTION

The disclosure provides a process for the integration of multiple processes, allowing the transformation of crude oil to multiple products with high olefin yield. We disclose an integrated process not including a steam cracker which is able to convert refinery C4, C5 and/or C6 streams to light olefins in the absence of steam cracking. Traditional FCC processes reduce reaction temperatures and therefore have lower energy consumption versus steam cracking, but do not yet provide high enough light olefin yields. Our disclosure integrates a catalytic naphtha cracking unit with an olefin paraffin separation unit and/or an olefin cracking unit by contacting a first feed stream comprising C4 and/or C5 olefins with a catalyst at first catalytic naphtha cracking conditions to produce a first cracked comprising ethylene and propylene.

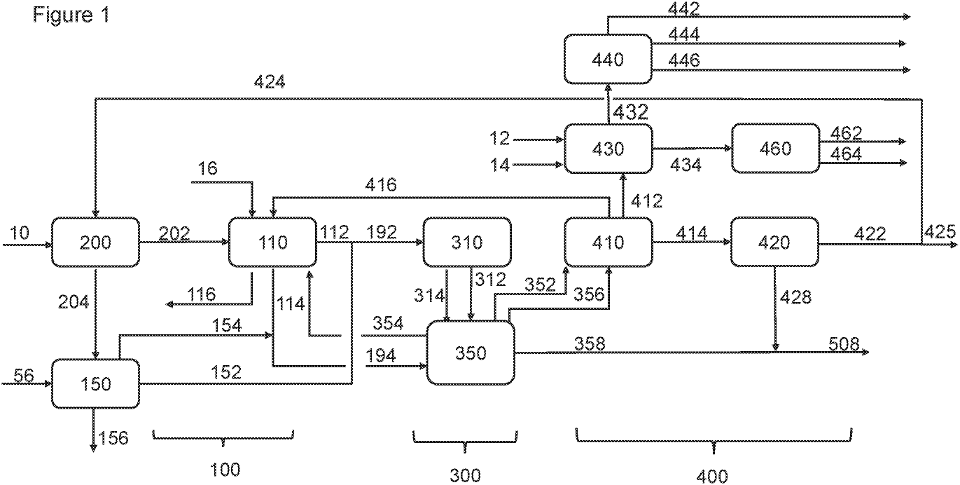

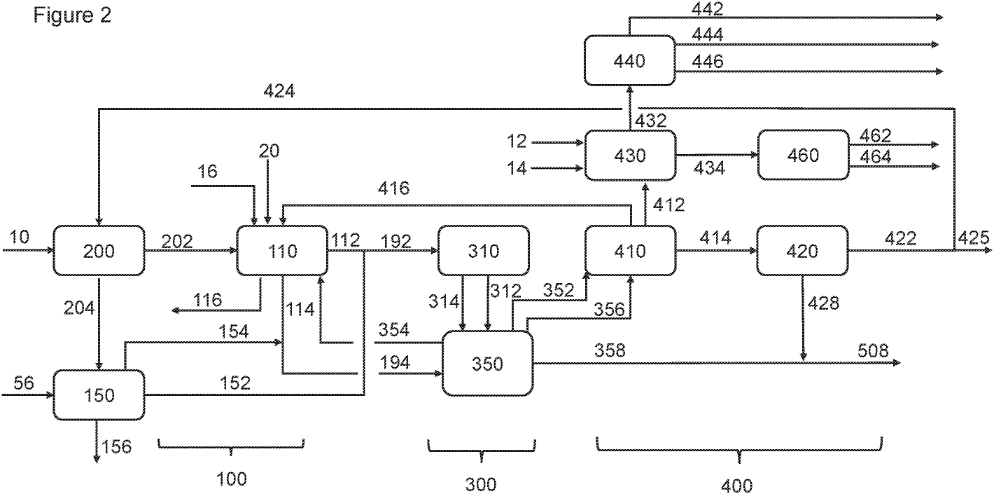

In an exemplary embodiment, a process feed stream comprising C4 and/or C5 hydrocarbons and possibly also comprising C6 hydrocarbons is fed to a catalytic naphtha cracking unit 100. Fluidized catalytic cracking processes combined with separation to yield a stream predominantly comprising C4 and/or C5 hydrocarbons may be an exemplary source of the process feed stream. Other refinery streams comprising C4 and/or C5 hydrocarbons may be contemplated. In a first embodiment shown in FIG. 1, the process feed stream in line 10 is first fed to an olefin paraffin separation unit 200. In a second embodiment shown in FIG. 2, the process feed stream in line 20 is fed to the first reactor unit 110 of the catalytic naphtha cracking unit 100. The remainder of FIG. 1 will now be described, with this description also applying to the remainder of FIG. 2.

In FIGS. 1 and 2, an olefin-paraffin separation unit 200 may be utilized to separate olefin species from paraffin species. From a feed stream comprising C4 hydrocarbons, butanes and other paraffin species such as n-butane and isobutane may be separated from butenes and other olefin species such as 1-butene and 2-butenes to form a first separated stream in line 202 rich in olefins and a second separated stream in line 204 rich in paraffins. Similar separation would occur for feed streams comprising C5 and/or C6 hydrocarbons. Cycloparaffins such as cyclopentanes may separate preferentially into the first separated stream in line 202 with olefinic species. The olefin-paraffin separation unit 200 may involve contacting a hydrocarbon predominantly comprising olefins and paraffins with suitable solvents, such as methylethylketone (MEK) or acetonitrile (ACN). or a suitable membrane at separation temperature and/or pressure. For ACN, suitable temperatures may range from about greater than 40° C. (104° F.) to less than about 140° C. (284° F.). Suitable temperatures may be greater than about 50° C. (122° F.) or less than about 110° C. (230° F.). Suitable pressures may be greater than 1 barg (14.5 psig) and less than 20 barg (290 psig). Pressure may be greater than 2 barg (29 psig) or less than about 10 barg (145 psig). Due to the different boiling points and/or different relative volatilities and/or the different diffusion properties of the paraffins and olefins, the paraffins will be predominantly separated from the olefins. The olefin-paraffin separation unit may utilize, but is not limited to, conventional distillation, extractive distillation, azeotropic distillation or membrane separation technology.

The olefin-paraffin separation unit 200 may produce a first separated stream comprising olefins, suitably rich in olefins, and preferably predominant in olefins, in line 202 and a second separated stream comprising paraffins, suitably rich in paraffins, and preferably predominant in paraffins, in line 204. The first separated stream in line 202 may predominantly comprise olefins. In an embodiment, the first separated stream comprises greater than 90 and preferably at least 95 wt% olefins. The second separated stream may predominantly comprise paraffins. The second separated stream may comprise greater than 80 and suitably at least 90 wt% paraffins.

Catalytic naphtha cracking involves contacting a hydrocarbon feed stream with a suitable cracking catalyst at reaction conditions to reduce the feed stream molecular weight by cracking and preferably produce light olefins defined here as ethylene and propylene. Larger paraffinic, naphthenic and larger olefinic molecules are cracked to smaller olefinic molecules while limiting aromatics formed. The contact may occur in a fixed bed, riser, or fluidized bed reactor at elevated temperature. The cracking reactions are endothermic, and heat may be supplied to the process by either heating the feed, catalyst, or both to suitable temperature before contact. In an embodiment, the catalytic naphtha cracking unit 100 may comprise two reactor units 150 and 110. The reactor units may have different reactor configuration, temperature, and catalyst depending on whether the feed stream predominantly comprises paraffins or olefins.

The catalyst in the catalytic naphtha cracking unit 100 may be a single catalyst or a mixture of different catalysts. Generally, the catalyst may comprise any of the well-known catalysts that are used in the art of FCC, such as an active amorphous clay-type catalyst and/or a high activity, crystalline molecular sieve. Zeolites may be used as molecular sieves in FCC processes. Preferably, the catalyst comprises a zeolite, an active alumina material, a binder material, and an inert filler such as kaolin. Collectively, the active alumina, binder material and inert filler may be known as matrix material.

The zeolite component may include a medium or smaller pore zeolite catalyst, such as a MFI zeolite, as exemplified by at least one of ZSM-5, ZSM-11, ZSM-12, ZSM-23, ZSM-35, ZSM-38, ZSM-48, and other similar materials. Other suitable medium or smaller pore zeolites include ferrierite, and erionite. These catalyst compositions may have a crystalline zeolite content of about 10 to about 50 wt-% or more, and a matrix material content of about 50 to about 90 wt-%. Components containing about 40 wt-% crystalline zeolite material are preferred, and those with greater crystalline zeolite content may be used. Generally, medium and smaller pore zeolites are characterized by having an effective pore opening diameter of less than or equal to about 0.7 nm and rings of about 10 or fewer members. Preferably, the zeolite component is an MFI zeolite having high silica to alumina ratios. Preferred silica to alumina ratios are greater than 300, with a more preferred ratio greater than 400, and a most preferred ratio greater than 1000. The catalyst crystals of the present invention may have the external surface acid site neutralized, thereby limiting the amount of activity on the external surface of the catalyst.

The first reactor unit 110 may be charged with a first separated stream in line 202 comprising olefins, suitably rich in olefins, and preferably predominant in olefins. A first steam supply in line 16 may be combined with the first separated stream 202 to atomize the feed to the first reactor unit 110. Steam is also used for fluidizing catalyst and purging in other parts of the first reactor unit 110. The first catalytic reactor 110, which may be a fixed bed, riser, or fluidized bed reactor, can operate at any suitable conditions, contacts hydrocarbon with a catalyst in the presence of steam to crack olefins in the first separated stream to smaller olefins.

The first catalytic reactor 110 typically operates at a temperature of about 400° C. (752° F.) to about 800° C. (1472° F.), preferably about 500° C. (932° F.) to about 620° C. (1148° F.), at the reactor outlet. Reactor outlet pressure may vary between about 20 psia (138.9 kPa) to about 75 psia (517.1 kPa), and more typically operates below 40 psia (275.8 kPa). Catalyst density in the first reactor unit 110 may be greater than 1 lb/ft3 (0.016 kg/L) and less than 20 lb/ft3 (0.32 kg/l). Catalyst to hydrocarbon (oil) ratio (C/O) based on the weight of catalyst and feed hydrocarbons entering the bottom of the reactor, may be in range from greater than 5 to less than 100. C/O may be greater than 20 or greater than 50. C/O may be less than 90 or less than 80. More typically the C/O may be between about 25:1 and 45:1. In an embodiment, the catalyst is the same as used in second reactor unit 150.

Following separation, which may comprise a quench tower and separator in which the reactor effluent is contacted with a water stream, the first reactor unit 110 may produce a first cracked gas stream in line 112, a first cracked liquid stream in line 114, and a first water stream in line 116. The first cracked gas stream may predominantly comprise hydrogen and C1-C5 hydrocarbons. The first cracked liquid stream may predominantly comprise C5+ hydrocarbons. The first cracked liquid stream may predominantly comprise C5+ olefins. The first water stream in line 116 may be reheated and recycled as a portion of the first steam supply in line 16.

The second reactor unit 150 may be charged with a second separated stream in line 204 comprising paraffins, suitably rich in paraffins, and preferably predominant in paraffins. A second steam supply in line 56 may be combined with the second separated stream in line 204 to form the feed to the second reactor unit 150. The second catalytic reactor unit 150, which may be a fixed bed, riser, or fluidized bed reactor, can operate at any suitable conditions, contacts hydrocarbon with a catalyst in the presence of steam to crack paraffins in the first separated stream to olefins. The second catalytic reactor unit 150 typically operates at a temperature of about 500° C. to about 800° C., preferably about 600° C. to about 650° C., at the reactor outlet. Reactor outlet pressure may vary between about 20 psia (138.9 kPa) to about 75 psia (517.1 kPa), and more typically operates below 40 psia (275.8 kPa). In an embodiment, the second reactor unit operates at a higher temperature than the first reactor unit.

Catalyst density in the first reactor unit 110 may be greater than 1 lb/ft3 (0.016 kg/L) and less than 20 lb/ft3 (0.32 kg/l). Catalyst to hydrocarbon (oil) ratio (C/O) based on the weight of catalyst and feed hydrocarbons entering the bottom of the reactor, may be in range from greater than 5 to less than 100. C/O may be greater than 20 or greater than 50. C/O may be less than 90 or less than 80. More typically the C/O may be between about 25:1 and 45:1. Steam in supply line 56 may be supplied into the second reactor 150 equivalent to about 0 to 75 wt% of feed. Typically, however, the steam rate may be between about 15% to about 60% for maximum light olefin production. The average vapor residence time in the reactor may be less than about 10 seconds.

Following separation, which may comprise a quench tower and separator in which the reactor effluent is contacted with a water stream, the second reactor unit 150 may produce a second cracked gas stream in line 152, a second cracked liquid stream in line 154 and a second water stream in line 156. The second cracked gas stream predominantly comprises hydrogen and C1-C5 hydrocarbons. The second cracked liquid stream may predominantly comprise C5+ hydrocarbons. The second cracked liquid stream may predominantly comprise C5+ olefins. The second water stream in line 156 may be reheated and recycled as a portion of the second steam supply in line 56.

The catalytic naphtha cracking unit may further comprise a regeneration zone. The regeneration zone may be a combined regeneration zone for both the first reaction unit 110 and the second reaction unit 150, or the first and second reaction units may have separate regeneration zones. The catalyst may be continuously or intermittently regenerated in the regenerator zone by burning off the coke, and fuel may be added in the regenerator combustion zone to achieve the desired heat balance.

In an embodiment, the second steam supply 56 and the first steam supply 16 form part of the same integrated steam supply system. In this embodiment, the second water stream in line 156 and the first water stream in line 116 may form part of the same integrated steam supply system. The second catalytic naphtha cracking reactor unit 150 and the first catalytic naphtha cracking reactor unit 110 may be in upstream and downstream communication with each other through the water lines 16, 56, 116, and 156 but not through the hydrocarbon feed lines.

In an embodiment, the second cracked gas stream in line 152 and the first cracked gas stream in line 112 may be combined to form a combined cracked gas stream in line 192 and fed to a compression unit 300. The second cracked liquid stream in line 154 and the first cracked liquid stream in line 114 may be combined to form a combined cracked liquid stream in line 194 and fed to a compression unit 300.

The compression unit 300 may comprise a first compression stage unit 310 and a second compression stage unit 350. The combined cracked gas stream in line 192 may be fed to the first compression stage unit 310. The first compression stage unit may involve using a multi-stage compressor to compress the combined cracked gas stream to an elevated pressure in the range from about 15 psia (103 kPa) to about 100 psia (689 kPa). The first compression stage unit may produce a first compressed gas stream in line 312 which predominantly comprises hydrogen and C1-C5 hydrocarbons. In an embodiment, the pressure may be from about 20 psia (138 kPa) to about 80 psia (551 kPa). The first compression stage unit 310 may produce a first compressed liquid stream in line 314 predominantly comprising C5+ hydrocarbons which may be fed to the second compression stage unit 350.

The combined cracked liquid stream in line 194 may be fed to the second compression stage unit 350. The combined cracked liquid stream and the first compressed liquid stream may be fed to the same stage, or different stages of the second compression stage unit 350. The second compression stage unit may involve using a multi-stage compressor to compress the cracked gas to an elevated pressure in the range from about 30 psia (206 kPa) to about 450 psia (3102 kPa). In an embodiment, the pressure may be from about 50 psia (345 kPa) to about 420 psia (2896 kPa). The second compression stage unit performs additional separation and may yield a second compressed gas stream in line 352 and a second compressed liquid stream in line 356. The second compressed gas stream may predominantly comprise hydrogen C2- hydrocarbons. The second compressed liquid stream may predominantly comprise C3-C5 hydrocarbons. The compressor in the first compression stage and the second compression stage may be different stages of the same compressor or separate compressors. The first compression stage unit 310 and the second compression stage unit 350 may be in upstream and downstream communication with each other.

The second compression stage unit 350 may further yield a third compressed gas stream in a recycle column side draw vapor line 354 and a third compressed liquid stream in a recycle column bottoms line 358. The third compressed liquid stream may comprise C6+ hydrocarbons and be purged from the process, possibly as a gasoline stream. The third compressed gas stream in line 354 may comprise C5 hydrocarbons and may be fed to the first catalytic naphtha cracking reactor unit 110.

A light olefin separation unit 400 is designed to separate the compressed streams into a plurality of product streams. A depropanizer unit 410 operated to separate C3- hydrocarbons from C4+ hydrocarbons may comprise a depropanizer column. The overhead pressure in a depropanizer column may be between about 2000 kPa (290 psig) and about 3500 kPa (508 psig) at an overhead temperature of between about 30° C. (86° F.) and about 80° C. (176° F.). Preferably, the overhead pressure in a depropanizer column may be between about 2200 kPa (319 psig) and about 3000 kPa (435 psig) at an overhead temperature of between about 40° C. (104° F.) and about 60° C. (140° F.). The bottom temperature in a depropanizer column may be between about 100° (212° F.) and about 200° C. (392° F.) and preferably between about 120° C. (248° F.) and about 180° C. (356° F.). The second compressed gas stream in line 352 and the second compressed liquid stream in line 356 may be fed to different tray locations of a depropanizer column in a depropanizer unit 410 and be separated into a first distilled stream 412 comprising C3- hydrocarbons and a second distilled stream 414 comprising C4+ hydrocarbons. A first distilled bottom stream in line 416 may also be produced.

The first distilled bottom stream in line 416 may comprise C3+ hydrocarbons or may comprise C4+ hydrocarbons or may comprise C5+ hydrocarbons. The first distilled bottom stream may be recycled to the catalytic naphtha cracking unit 100. Preferably, the first distilled bottom stream is recycled to the first reactor unit 110.

The first distilled stream 412 may be fed to a deethanizer unit 430 along with a first FCC stream in line 12 comprising C2- hydrocarbons and a second FCC stream in line 14 comprising C3 hydrocarbons. A deethanizer unit 430 operated to separate C2- hydrocarbons from C3+ hydrocarbons may comprise a deethanizer column. The overhead pressure in a deethanizer column may be between about 1800 kPa (261 psig) and about 2600 kPa (377 psig) at an overhead temperature of between about -60° C. (-76° F.) and about -20° C. (-4° F.). Preferably, the overhead pressure in a deethanizer column may be between about 2000 kPa (290 psig) and about 2400 kPa (348 psig) at an overhead temperature of between about -50° C. (-58° F.) and about -30° C. (-22° F.). The bottom temperature in a deethanizer column may be between about 10° (50° F.) and about 100° C. (212° F.) and preferably between about 30° C. (86° F.) and about 80° C. (176° F.). In an embodiment, the first distilled stream may be combined with the first FCC stream and the second FCC stream to form a combined C2-C3 product stream prior to feeding the deethanizer unit 430. The deethanizer unit 430 may produce a third distilled stream in line 432 comprising C2- hydrocarbons and a fourth distilled stream in line 434 comprising C3 hydrocarbons.

The third distilled stream in line 432 may be fed to an ethylene recovery unit 440 (ERU) which recovers ethylene, ethane, C2- hydrocarbons and hydrogen and may comprise pressure swing adsorption and/or cryogenic distillation and/or membrane separation for further separation. The ERU may produce a fuel gas stream in line 442, an ethylene stream in line 444 and an ethane stream in line 446. The ethylene stream may predominantly comprise ethylene, or may comprise greater than 90% ethylene, or may comprise greater than 95% ethylene or may comprise greater than 99% ethylene or may comprise greater than 99.5% ethylene.

The fourth distilled stream in line 434 may be fed to a propane-propylene separation unit 460 for separation into a propylene stream in line 462 and a propane stream in line 464. The propane-propylene separation unit 460 may comprise a propane-propylene (PP) splitter column, a membrane separation unit, or other technology known to separate propane from propylene. The overhead pressure in a PP splitter column may be between about 400 kPa (58 psig) and about 1200 kPa (174 psig) at an overhead temperature of between about 0° C. (32° F.) and about 60° C. (140° F.). Preferably, the overhead pressure in a PP splitter column may be between about 600 kPa (87 psig) and about 1000 kPa (145 psig) at an overhead temperature of between about 10° C. (50° F.) and about 50° C. (122° F.). The bottom temperature in the PP splitter column may be between about 0° (32° F.) and about 60° C. (140° F.) and preferably between about 10° C. (50° F.) and about 50° C. (122° F.). The propylene stream in line 462 may predominantly comprise propylene, or may comprise greater than 90% propylene, or may comprise greater than 95% propylene or may comprise greater than 99% propylene or may comprise greater than 99.5% propylene.

The second distilled stream 414 may be fed to a dehexanizer unit 420 for further separation. A dehexanizer unit 420 operated to separate C5- hydrocarbons from C6+ hydrocarbons may comprise a dehexanizer column. The overhead pressure in a dehexanizer column may be between about 500 kPa (72 psig) and about 1500 kPa (218 psig) at an overhead temperature of between about 10° C. (50° F.) and about 80° C. (176° F.). Preferably, the overhead pressure in a dehexanizer column may be between about 800 kPa (116 psig) and about 1200 kPa (174 psig) at an overhead temperature of between about 20° C. (68° F.) and about 70° C. (178° F.). The bottom temperature in a dehexanizer column may be between about 100° (212° F.) and about 300° C. (572° F.) and preferably between about 150° C. (302° F.) and about 250° C. (482° F.). The dehexanizer unit 420 may produce a fifth distilled stream in line 422 predominantly comprising C4 and/or C5 hydrocarbons, and a sixth distilled stream in line 428 comprising C6+ hydrocarbons. The fifth distilled stream may comprise olefins but will predominantly comprise paraffins. The sixth distilled stream in line 428 may be combined with the compressed liquid stream in bottoms line 358 forming a combined C6+ hydrocarbon stream in line 508 to be purged from the process or used as a gasoline stream.

An aliquot, or all of the fifth distilled stream, may be utilized as a recycle product stream in line 424. A remaining aliquot of the fifth distilled stream may be purged from the system in line 425 or used as a gasoline stream. The recycle product stream in line 424 may be fed to the olefin-paraffin separation unit 200 or catalytic naphtha cracking unit 100.

The light olefin separation unit 400 may be in upstream and downstream communication with the olefin-paraffin separation unit 200 and the catalytic naphtha cracking unit 100.

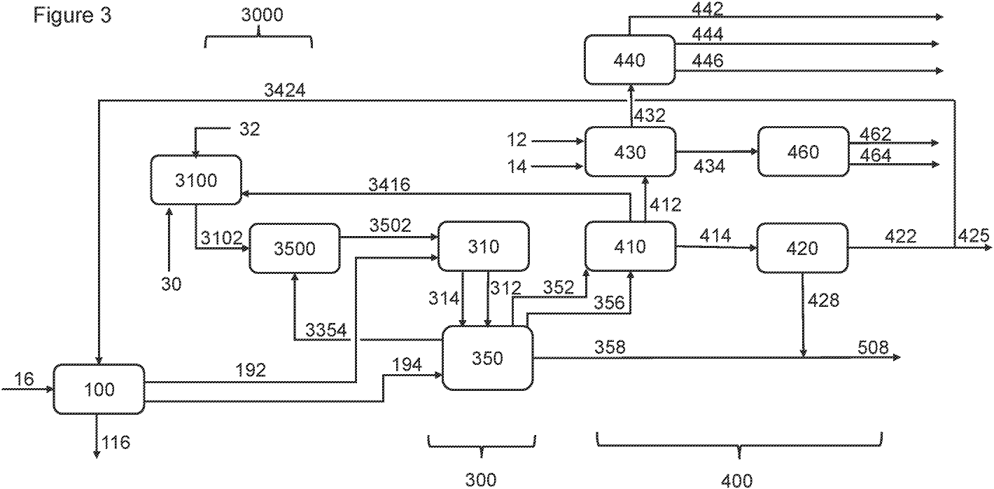

In an alternate embodiment shown in FIG. 3, the process feed stream in line 30 is first fed to an olefin cracking process unit 3000. In the alternate embodiment, an olefin-paraffin separation unit 200 has been removed, and an olefin cracking unit process unit 3000 used to convert olefin species. A recycle product stream continues to be fed to the catalytic naphtha cracking unit 100. Elements of FIG. 3 not described here are as described for the embodiment in FIG. 1.

An olefin cracking process unit converts larger olefins, C4+ hydrocarbons, to light olefins of ethylene and propylene, but primarily propylene. The production of light olefins from the olefin cracking process unit 3000 does not consume ethylene, and the overall conversion of larger olefins to ethylene and propylene may be about 60%, with a reasonable amount of recycle. The olefin cracking process unit 3000 may also produce butenes when there is a significant amount of C5+ olefins in the feed. The olefin cracking process unit 3000 may comprise a butadiene selective conversion zone 3100 to convert dienes to mono-olefins.

Hydrogen in line 32 may optionally be added to the process feed stream in line 30 before it is fed, or be fed separately, to the butadiene selective conversion zone 3100 for selective hydrogenation of dienes to form a treated process feed stream in line 3102 rich in olefins and lean in dienes.

The butadiene conversion zone 3100 is normally operated at relatively mild hydrogenation conditions in the liquid phase. A broad range of suitable operating pressures in the butadiene conversion zone range from about 276 kPag (40 psig) to about 5516 kPag (800 psig), or about 345 kPag (50 psig) to about 2069 kPag (300 psig). A relatively moderate temperature between about 25° C. (77° F.) and about 350° C. (662° F.), or about 50° C. (122° F.) to about 200° C. (392° F.) is typically employed. The liquid hourly space velocity of the reactants for the selective hydrogenation catalyst may be above about 1.0 hr-1, or above about 10 hr-1, or above about 30 hr-1, to about 50 hr-1. To avoid the undesired saturation of a significant amount mono-olefinic hydrocarbons, the mole ratio of hydrogen to multi-olefinic hydrocarbons in the material entering the bed of selective hydrogenation catalyst is maintained between 0.75:1 and 1.8:1. A selective hydrogenation catalyst is used for the butadiene conversion in the process feed stream in line 30. A selective hydrogenating catalyst may be any suitable catalyst which is capable of selectively hydrogenating butadiene in a C4 stream may be used in the present disclosure. A particularly preferred selective hydrogenation catalyst comprise copper and at least one other metal such as titanium, vanadium, chrome, manganese, cobalt, nickel, zinc, molybdenum, and cadmium or mixtures thereof. The metals are preferably supported on inorganic oxide supports such as silica and alumina. Preferably, a selective hydrogenation catalyst may comprise a copper and a nickel metal supported on alumina.

The olefin cracking process unit 3000 may comprise an olefin cracking reaction unit 3500. Catalysts suitable for olefin cracking reaction comprise a crystalline silicate of the MFI family which may be a zeolite, a silicalite or any other silicate in that family or the MEL family which may be a zeolite or any other silicate in that family. Examples of MFI silicates are ZSM-5 and silicalite. An example of an MEL zeolite is ZSM-11 which is known in the art. Other examples are Boralite D and silicalite-2 as described by the International Zeolite Association (ATLAS OF ZEOLITE STRUCTURE TYPES, 1987, Butterworths). The preferred crystalline silicates have pores or channels defined by ten oxygen rings and a high silicon/aluminum atomic ratio.

The crystalline silicate catalyst has structural and chemical properties and is employed under particular reaction conditions whereby the catalytic cracking of the C4 to C7 olefins readily proceeds. Different reaction pathways can occur on the catalyst. Suitable olefin cracking process conditions include an inlet temperature of around 400° C. to 600° C., preferably from 520° C. to 600° C., yet more preferably 540° C. to 580° C., and an olefin partial pressure of from 10 to 202 kPa absolute (1.5 to 29 psia), preferably from 50 to 152 kPa absolute (7 to 22 psia).

A crystalline silicate catalyst possessing a high silicon/aluminum ratio can achieve a stable olefin conversion with a high propylene yield on an olefin basis of from 20 to 50 wt-% with the olefinic feedstocks of the present invention. The MFI catalyst having a high silicon/aluminum atomic ratio for use in the catalytic olefin cracking process of the present invention may be manufactured by removing aluminum from a commercially available crystalline silicate as is known in the art. The commercially available MFI crystalline silicate may be modified by processes comprising steaming which reduces the tetrahedral aluminum in the crystalline silicate framework and converts the aluminum atoms into octahedral aluminum in the form of amorphous alumina. The framework silicon/aluminum ratio may be increased by this process to a value of at least about 180, preferably from about 180 to 1000, more preferably at least 200, yet more preferably at least 300 and most preferably around 480.

The MEL or MFI crystalline silicate catalyst may be mixed with a binder, preferably an inorganic binder, and shaped to a desired shape; e.g., extruded pellets. The binder is selected so as to be resistant to the temperature and other conditions employed in the catalyst manufacturing process and in the subsequent catalytic cracking process for the olefins. The binder is an inorganic material selected from clays, silica, metal oxides such as ZrO2 and/or metals, or gels including mixtures of silica and metal oxides. The binder is preferably alumina-free, although aluminum in certain chemical compounds as in AlPO4 may be used as the latter are quite inert and not acidic in nature. If the binder which is used in conjunction with the crystalline silicate is itself catalytically active, this may alter the conversion and/or the selectivity of the catalyst. Inactive materials for the binder may suitably serve as diluents to control the amount of conversion, so that products can be obtained economically and orderly without employing other means for controlling the reaction rate. It is desirable to provide a catalyst having a good crush strength to prevent the catalyst from breaking down into powder-like materials during use. Such clay or oxide binders have been employed normally for the purpose of improving the crush strength of the catalyst. A particularly preferred binder for the catalyst of the present invention comprises silica or AlPO4.

The relative proportions of the finely divided crystalline silicate material and the inorganic oxide matrix of the binder can vary widely. Typically, the binder content ranges from 5 to 95% by weight, more typically from 20 to 50% by weight, based on the weight of the composite catalyst. Such a mixture of crystalline silicate and an inorganic oxide binder is referred to as a formulated crystalline silicate.

In mixing the catalyst with a binder, the catalyst may be formulated into pellets, spheres, extruded into other shapes, or formed into a spray-dried powder. In the olefin cracking process, the process conditions are selected in order to provide high selectivity towards propylene or ethylene, as desired, a stable olefin conversion over time, and a stable olefinic product distribution in the effluent. Such objectives are favored by the use of a low acid density in the catalyst (i.e., a high Si/Al atomic ratio) in conjunction with a low pressure, a high inlet temperature and a short contact time, all of which process parameters are interrelated and provide an overall cumulative effect.

The process conditions in the olefin cracking reaction unit 3500 are selected to disfavor hydrogen transfer reactions leading to the formation of paraffins, aromatics and coke precursors. The process operating conditions thus employ a high space velocity, a low pressure and a high reaction temperature. The LHSV ranges from 5 to 30 hr-1, preferably from 10 to 30 hr-1. The olefin partial pressure ranges from 10 to 202 kPa absolute (1.5 to 29 psia), preferably from 50 to 152 kPa absolute (7 to 22 psia). A particularly preferred olefin partial pressure is atmospheric pressure. The hydrocarbon feedstocks are preferably fed at a total inlet pressure sufficient to convey the feedstocks through the reactor. The hydrocarbon feedstocks may be fed undiluted or diluted in an inert gas; e.g., nitrogen or steam. The total absolute pressure in the reactor ranges from 30 to 1013 kPa absolute (4 to 147 psia) and is preferably atmospheric. The use of a low olefin partial pressure, for example atmospheric pressure, tends to lower the incidence of hydrogen transfer reactions in the cracking process, which in turn reduces the potential for coke formation which tends to reduce catalyst stability. The cracking of the olefins is preferably performed at an inlet temperature of the feedstock of from 400° C. to 650° C., more preferably from 450° C. to 600° C., yet more preferably from 540° C. to 590° C., typically around 560° C. to 585° C.

The olefin cracking reaction unit 3500 may produce a third cracked gas stream comprising C5- hydrocarbons and rich in olefins in line 3502. The compression unit 300 operates as described for FIG. 1. The third cracked gas stream may be fed to the first compression stage unit 310, either combined with, or separately from the combined cracked gas stream in line 192. The third compressed gas stream in line 3354 may comprise C5 hydrocarbons and may be fed to the olefin cracking process unit 3000, preferably to the olefin cracking reaction unit 3500. The first compression stage unit 310 may be in upstream and downstream communication with the olefin cracking reaction unit 3500.

In this embodiment, the first distilled bottom stream in line 3416 may comprise C3+ hydrocarbons or may comprise C4+ hydrocarbons or may comprise C5+ hydrocarbons. The first distilled bottom stream may be recycled to the olefin cracking process unit 3000. Preferably, the first distilled bottom stream is recycled to the butadiene selective conversion zone 3100.

In this embodiment, the recycle product stream in line 3424 which may predominantly comprise paraffins may be fed to the catalytic naphtha cracking unit 100. The light olefin separation unit 400 may be in upstream and downstream communication with the catalytic naphtha cracking unit 100 and the olefin cracking process unit 3000.

### SPECIFIC EMBODIMENTS

While the following is described in conjunction with specific embodiments, it will be understood that this description is intended to illustrate and not limit the scope of the preceding description and the appended claims.

A first embodiment of the disclosure is a process for producing ethylene and propylene, comprising contacting a first feed stream comprising C4 and/or C5 olefins with a catalyst at first catalytic naphtha cracking conditions to produce a first cracked product, compressing the cracked product to a higher pressure, separating the compressed cracked product into a plurality of product streams, recovering a product stream comprising ethylene and a product stream comprising propylene, and recycling at least a portion of a product stream comprising C4 and C5 olefins to be contacted with the catalyst as a portion of the first feed stream. An embodiment of the disclosure is one, any or all of prior embodiments in this paragraph up through the first embodiment in this paragraph further comprising contacting a stream comprising C4 and C5 hydrocarbons with an olefin-paraffin separation unit to produce a first separated stream comprising C4 and C5 olefins and a second separated stream comprising C4 and C5 paraffins, wherein the first separated stream forms at least a part of the first feed stream. An embodiment of the disclosure is one, any or all of prior embodiments in this paragraph up through the first embodiment in this paragraph wherein the compression is to a pressure of from about 103 kPa (15 psia) to about 689 kPa (100 psia). An embodiment of the disclosure is one, any or all of prior embodiments in this paragraph up through the first embodiment in this paragraph further comprising contacting the first feed stream with the catalyst in the presence of steam. An embodiment of the disclosure is one, any or all of prior embodiments in this paragraph up through the first embodiment in this paragraph further comprising contacting the second separated stream with a catalyst at second catalytic naphtha cracking conditions to produce a second cracked product. An embodiment of the disclosure is one, any or all of prior embodiments in this paragraph up through the first embodiment in this paragraph wherein the first feed stream further comprises at least a portion of C4 and C5 hydrocarbons derived from an FCC unit. An embodiment of the disclosure is one, any or all of prior embodiments in this paragraph up through the first embodiment in this paragraph further comprising distilling the compressed cracked product to form a first distilled stream comprising C3- hydrocarbons, a second distilled stream comprising C4+ hydrocarbons, and combining the first distilled stream with a first FCC stream comprising C2- hydrocarbons and a second FCC stream comprising C3 hydrocarbons to form a combined C2-C3 product stream prior to recovering the product stream comprising ethylene. An embodiment of the disclosure is one, any or all of prior embodiments in this paragraph up through the first embodiment in this paragraph further comprising distilling the combined C2-C3 product stream to form a third distilled stream comprising C2- hydrocarbons and a fourth distilled stream comprising C3 hydrocarbons. An embodiment of the disclosure is one, any or all of prior embodiments in this paragraph up through the first embodiment in this paragraph further comprising recovering the product stream comprising propylene from the fourth distilled stream. An embodiment of the disclosure is one, any or all of prior embodiments in this paragraph up through the first embodiment in this paragraph further comprising recycling at least a portion of the second distilled stream to be contacted with the catalyst at catalytic cracking conditions as a portion of the first feed stream. An embodiment of the disclosure is one, any or all of prior embodiments in this paragraph up through the first embodiment in this paragraph further comprising contacting the second feed stream with the catalyst in the presence of steam. An embodiment of the disclosure is one, any or all of prior embodiments in this paragraph up through the first embodiment in this paragraph wherein first catalytic naphtha cracking conditions include a temperature of from about 400° C. (752° F.) to about 800° C. (1472° F.), a reactor outlet pressure of from about 20 psia (138.9 kPa) to about 75 psia (517.1 kPa), a catalyst density of greater than 1 lb/ft3 (0.016 kg/L) and less than 20 lb/ft3 (0.32 kg/l), and a C/O of from greater than 5 to less than 100. An embodiment of the disclosure is one, any or all of prior embodiments in this paragraph up through the first embodiment in this paragraph wherein second catalytic naphtha cracking conditions include a temperature of from about 500° C. (932° F.) to about 800° C. (1472° F.), a reactor outlet pressure of from about 20 psia (138.9 kPa) to about 75 psia (517.1 kPa), a catalyst density of greater than 1 lb/ft3 (0.016 kg/L) and less than 20 lb/ft3 (0.32 kg/l), and a C/O of from greater than 5 to less than 100. An embodiment of the disclosure is one, any or all of prior embodiments in this paragraph up through the first embodiment in this paragraph wherein second catalytic naphtha cracking conditions include a temperature higher temperature than first catalytic naphtha cracking conditions.

A second embodiment of the disclosure is a process for producing ethylene and propylene, comprising contacting a first feed stream comprising C4 and C5 olefins with a catalyst at first catalytic naphtha cracking conditions to produce a first cracked product, contacting a second feed stream comprising C4 and C5 paraffins with a catalyst at second catalytic naphtha cracking conditions to produce a second cracked product, compressing both cracked products to a higher pressure, separating the compressed cracked product into a plurality of product streams, recovering a product stream comprising ethylene and/or a product stream comprising propylene, and recycling at least a portion of a product stream comprising C4 and C5 olefins to be contacted with the catalyst as a portion of the first feed stream. An embodiment of the disclosure is one, any or all of prior embodiments in this paragraph up through the second embodiment in this paragraph further comprising contacting the product stream comprising C4 and C5 olefins with an olefin-paraffin separation unit to produce a first separated stream comprising C4 and C5 olefins and a second separated stream comprising C4 and C5 paraffins, wherein the first separated stream forms at least a part of the first feed stream. An embodiment of the disclosure is one, any or all of prior embodiments in this paragraph up through the second embodiment in this paragraph wherein the second separated stream forms at least a part of the second feed stream. An embodiment of the disclosure is one, any or all of prior embodiments in this paragraph up through the second embodiment in this paragraph wherein first catalytic naphtha cracking conditions include a temperature of from about 400° C. (752° F.) to about 800° C. (1472° F.), a reactor outlet pressure of from about 20 psia (138.9 kPa) to about 75 psia (517.1 kPa), a catalyst density of greater than 1 lb/ft3 (0.016 kg/L) and less than 20 lb/ft3 (0.32 kg/l), and a C/O of from greater than 5 to less than 100. An embodiment of the disclosure is one, any or all of prior embodiments in this paragraph up through the second embodiment in this paragraph wherein second catalytic naphtha cracking conditions include a temperature of from about 500° C. (932° F.) to about 800° C. (1472° F.), a reactor outlet pressure of from about 20 psia (138.9 kPa) to about 75 psia (517.1 kPa), a catalyst density of greater than 1 lb/ft3 (0.016 kg/L) and less than 20 lb/ft3 (0.32 kg/l), and a C/O of from greater than 5 to less than 100.

A third embodiment of the disclosure is a process for producing ethylene and propylene, comprising contacting a first feed stream comprising C4 and C5 olefins with a catalyst at catalytic naphtha cracking conditions to produce a first cracked product, contacting a second feed stream comprising C4 and C5 olefins with a catalyst at olefin cracking conditions to produce a second cracked product, compressing both cracked products to a higher pressure, separating the compressed cracked product into a plurality of product streams, recovering a product stream comprising ethylene and a product stream comprising propylene, and recycling a product stream comprising C4 and C5 olefins to be contacted with the catalyst as a portion of the first feed stream.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. An integrated process for producing ethylene and propylene, comprising:
contacting a stream comprising C4 and C5 hydrocarbons with an olefin-paraffin separation unit to produce a first separated stream comprising C4 and C5 olefins and a second separated stream comprising C4 and C5 paraffins, wherein the first separated stream comprises at least 75 wt% olefins,
contacting the first separated stream comprising C4 and C5 olefins with a catalyst at first catalytic naphtha cracking conditions to produce a first cracked product,
compressing the first cracked product to a higher pressure to produce a compressed cracked product,
separating the compressed cracked product into a plurality of product streams,
recovering a product stream comprising ethylene and a product stream comprising propylene, and
recycling at least a portion of a product stream comprising C4 and C5 olefins to be contacted with the catalyst as a portion of the first feed stream.

2. (canceled)

3. The process of claim 1 wherein the compression is to a pressure of from about 103 kPa (15 psia) to about 689 kPa (100 psia).

4. The process of claim 1 further comprising contacting the first feed stream with the catalyst in the presence of steam.

5. The process of claim 2 further comprising contacting the second separated stream with a catalyst at second catalytic naphtha cracking conditions to produce a second cracked product.

6. The process of claim 1 wherein the first feed stream further comprises at least a portion of C4 and C5 hydrocarbons derived from an FCC unit.

7. The process of claim 1 further comprising
distilling the compressed cracked product to form a first distilled stream comprising C3-hydrocarbons, a second distilled stream comprising C4+ hydrocarbons, and
combining the first distilled stream with a first FCC stream comprising C2- hydrocarbons and a second FCC stream comprising C3 hydrocarbons to form a combined C2-C3 product stream prior to recovering said product stream comprising ethylene.

8. The process of claim 7 further comprising distilling the combined C2-C3 product stream to form a third distilled stream comprising C2- hydrocarbons and a fourth distilled stream comprising C3 hydrocarbons.

9. The process of claim 8 further comprising recovering the product stream comprising propylene from the fourth distilled stream.

10. The process of claim 7 further comprising recycling at least a portion of the second distilled stream to be contacted with the catalyst at catalytic cracking conditions as a portion of the first feed stream.

11. The process of claim 5 further comprising contacting the second separated stream with the catalyst in the presence of steam.

12. The process of claim 1 wherein first catalytic naphtha cracking conditions include a temperature of from about 400° C. (752° F.) to about 800° C. (1472° F.), a reactor outlet pressure of from about 20 psia (138.9 kPa) to about 75 psia (517.1 kPa), a catalyst density of greater than 1 lb/ft3 (0.016 kg/L) and less than 20 lb/ft3 (0.32 kg/l), and a C/0 of from greater than 5 to less than 100.

13. The process of claim 5 wherein second catalytic naphtha cracking conditions include a temperature of from about 500° C. (932° F.) to about 800° C. (1472° F.), a reactor outlet pressure of from about 20 psia (138.9 kPa) to about 75 psia (517.1 kPa), a catalyst density of greater than 1 lb/ft3 (0.016 kg/L) and less than 20 lb/ft3 (0.32 kg/l), and a C/0 of from greater than 5 to less than 100.

14. The process of claim 13 wherein second catalytic naphtha cracking conditions include a temperature higher temperature than first catalytic naphtha cracking conditions.

15. An integrated process for producing ethylene and propylene, comprising:
contacting a first feed stream comprising C4 and C5 olefins with a catalyst at first catalytic naphtha cracking conditions to produce a first cracked product,
contacting a second feed stream comprising C4 and C5 paraffins with a catalyst at second catalytic naphtha cracking conditions to produce a second cracked product,
compressing both cracked products to a higher pressure,
separating the compressed cracked product into a plurality of product streams,
recovering a product stream comprising ethylene and/or a product stream comprising propylene, and
recycling at least a portion of a product stream comprising C4 and C5 olefins to be contacted with the catalyst as a portion of the first feed stream.

16. The process of claim 15 further comprising contacting the product stream comprising C4 and C5 olefins with an olefin-paraffin separation unit to produce a first separated stream comprising C4 and C5 olefins and a second separated stream comprising C4 and C5 paraffins,
wherein the first separated stream forms at least a part of the first feed stream.

17. The process of claim 16 wherein the second separated stream forms at least a part of the second feed stream.

18. The process of claim 15 wherein first catalytic naphtha cracking conditions include a temperature of from about 400° C. (752° F.) to about 800° C. (1472° F.), a reactor outlet pressure of from about 20 psia (138.9 kPa) to about 75 psia (517.1 kPa), a catalyst density of greater than 1 lb/ft3 (0.016 kg/L) and less than 20 lb/ft3 (0.32 kg/l), and a C/0 of from greater than 5 to less than 100.

19. The process of claim 15 wherein second catalytic naphtha cracking conditions include a temperature of from about 500° C. (932° F.) to about 800° C. (1472° F.), a reactor outlet pressure of from about 20 psia (138.9 kPa) to about 75 psia (517.1 kPa), a catalyst density of greater than 1 lb/ft3 (0.016 kg/L) and less than 20 lb/ft3 (0.32 kg/l), and a C/0 of from greater than 5 to less than 100.

20. An integrated process for producing ethylene and propylene, comprising:
contacting a first feed stream comprising C4 and C5 olefins with a catalyst at catalytic naphtha cracking conditions to produce a first cracked product,
contacting a second feed stream comprising C4 and C5 olefins with a catalyst at olefin cracking conditions to produce a second cracked product,
compressing both cracked products to a higher pressure,
separating the compressed cracked product into a plurality of product streams,
recovering a product stream comprising ethylene and a product stream comprising propylene, and
recycling a product stream comprising C4 and C5 olefins to be contacted with the catalyst as a portion of the first feed stream.
